(19) United States
(12) Patent Application Publication
(10) Pub. No.: US 2016/0128577 A1
(43) Pub. Date: May 12, 2016

(54) System for Measuring Skin Hydration

(71) Applicant: Nova Technology Corporation, Meredith, NH (US)

(72) Inventors: Michael J.E. Campbell, Meredith, NH (US); Merchon G. Cottrell, Merrimack, NH (US); Charles A. Huckins, Pembroke, NH (US)

(21) Appl. No.: 14/997,641

(22) Filed: Jan. 18, 2016

Related U.S. Application Data
(63) Continuation of application No. 14/013,743, filed on Aug. 29, 2013.
(60) Provisional application No. 61/696,147, filed on Sep. 1, 2012.

(51) Int. Cl. A61B 5/00; A61B 5/053

(52) U.S. Cl. CPC A61B 5/0077; A61B 5/0531; A61B 5/0537; A61B 5/72; A61B 5/7475; A61B 5/742; A61B 5/0022

(57) ABSTRACT

A system for measuring skin hydration at a measurement site with a independent measurement probe and a portable data processing system. Wireless communications enable the transfer of commands and data between the probe and portable data processing system. The portable data processing system can comprise a wireless tablet or smartphone. The portable data processing system provides input functions and output functions on its display and includes a camera for imaging the measurement site.

### CROSS-REFERENCE TO RELATED APPLICATIONS

This application is a conversion of co-pending U.S. patent application Ser. No. 14/016,743 filed Sep. 3, 2013 for a System for Measuring Skin Hydration which is a conversion of Provisional Application Ser. No. 61/696,147 filed Sep. 1, 2012 for a System for Measuring Skin Hydration.

### BACKGROUND OF THE INVENTION

1. Field of the Invention

This invention generally relates to the measurement of skin hydration and more specifically to systems that facilitate diagnoses in which dermal phase meters provide data useful in measuring skin hydration.

2. Description of Related Art

Over the years there has been a growing interest in measuring the relative hydration of a patient's skin at a diagnostic site for determining certain biophysical characteristics of that site. U.S. Pat. No. 5,961,471 to Nickson discloses a probe for biophysical skin measurements that includes a handle that carries a disposable sensor and for receiving a cable from a measurement device that produces data transferred to data processing system over a cable. In use, a diagnostician must manipulate or position each of the probe, measurement device and data processing system prior to making a measurement and during the measurement.

U.S. Pat. No. 6,370,426 to Campbell et al. and U.S. Pat. No. 7,219,534 to Campbell disclose other apparatus for measuring the relative hydration of a substrate, such as the skin. This apparatus combines the sensor and measurement device in a single unit. A cable connects this single unit to a system control that measures electrical characteristics and temperature of the substrate or skin and the force applied to the substrate to provide data for determining a relative hydration. The diagnostician interfaces with a system control in the form of a data processing system. Although this system reduces the number of pieces of apparatus, the diagnostician may still be required to manipulate the position of a probe and the data processing system.

A diagnosis involving a measurement of skin hydration additionally may involve information obtained by visual inspection of the area being tested during a number of measurement sessions. In some situations this information is recorded in the form of a written note or description that may be stored in hard copy form in a patient's medical file or in the form of recorded oral statements stored in analog or digital form for each session. In either form, the information about the appearance of the area being tested is subjective and may not be consistent if different measurement sessions are conducted by different diagnosticians. It is also possible to image the area being tested during each session. However, such images require imaging equipment, such as cameras, and a protocol for storing those images. All of this has complicated diagnostic protocols for measuring skin hydration.

### SUMMARY

Therefore, it is an object of this invention to provide a system that can simplify diagnostic protocols for measuring skin hydration.

Another object of this invention to provide a system that can simplify diagnostic protocols for measuring skin hydration with a dermal phase meter

Still another object of this invention is to provide a system that facilitates skin hydration measurements by enabling the storage of measurement data and images of a measurement area in a conveniently retrievable and displayable manner.

In accordance with this invention, a system for measuring skin hydration comprises a probe for measuring skin impedance at a measurement site including an independently powered measurement control for obtaining measurements in response to measurement commands and for generating information including the outcome of the skin impedance measurement. A portable data processing system generates the measurement commands and receives measurement information generated said probe. The data processing system has an operator interface and visual display. A wireless communications network interconnects the probe and the portable data processing system whereby skin impedance measurements can be displayed at the portable data processing system.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 depicts, in schematic form, a system for measuring skin hydration that incorporates this invention; and

FIG. 2 is a flow chart that depicts the operation of the system shown in FIG. 1.

### DESCRIPTION OF AN ILLUSTRATIVE EMBODIMENT

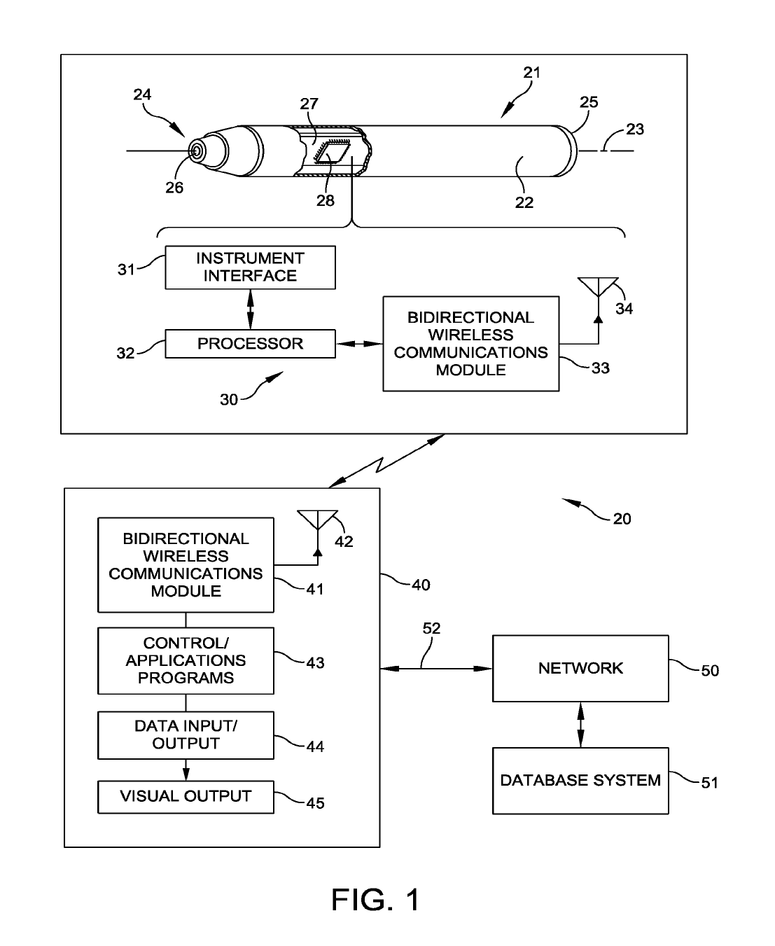

FIG. 1 depicts one embodiment of a skin hydration measurement system 20 that includes an independent measurement probe 21 that is particularly useful in measuring skin hydration at a measurement area, such as a burn location. The probe 21 has a cylindrical housing 22 lying along an axis 23 between a first, distal end 24 and a second, proximal end 25. In the case of the probe 21, “distal end” is meant to refer to the end that is most proximate the individual being undergoing a measurement. The probe housing 22 supports a sensor body 26 at the distal end 24 and houses a printed circuit board 27. The printed circuit board 27 carries a number of components, one such component being represented by an integrated circuit 28. The printed circuit board 27 and its components (not shown) enable the probe 21 to be operated as a self-contained, battery-operated device.

Still referring to FIG. 1, functionally the printed circuit board 27 includes an associated control 30 that responds to high level input commands by generating measurement signals based upon the skin impedance at the sensor 26. More specifically, the control 30 includes an instrument interface 31 and a processor 32. The interface 31 and processor 32 provide a means for controlling the operation of the probe 21 in response to high level commands and for receiving data in the form of signals from the probe 21 during a measurement session. In accordance with this invention, the processor 32 connects to a bidirectional wireless communications module 33, such as a Bluetooth® wireless telecommunication module with a transmit-receive antenna 34 or functionally equivalent module.

During a measurement session, the accumulated data from the measurement transfers to a portable data processing system 40 is transmitted by the module 33 and antenna 34 for receipt by means of a bidirectional wireless communications module 41 with a transmit-receive antenna assembly 42. A control/applications programs module 43, a data input/output module 44 and a visual output module 45 all operate to generate sequences of high-level commands to and to receive measurement data from the control 30. The system 40 may comprise a wireless tablet (e.g., an Apple® iPad ® tablet) or a smart phone (e.g., an Apple iPhone® mobile telephone) that allows the diagnostician to interact with the system 40. As known, such tablets and telephones include a camera as an imaging system, not shown, that enable the diagnostician to record one or more images of the measurement area as part of the diagnostic protocol without having to handle additional equipment.

Wireless or hardwire connections are also possible between the portable data processing system 40 and a network 50 and a database system 51 or other communications path. Such a configuration enables each diagnostic session to be recorded and stored for later retrieval when needed from a central site.

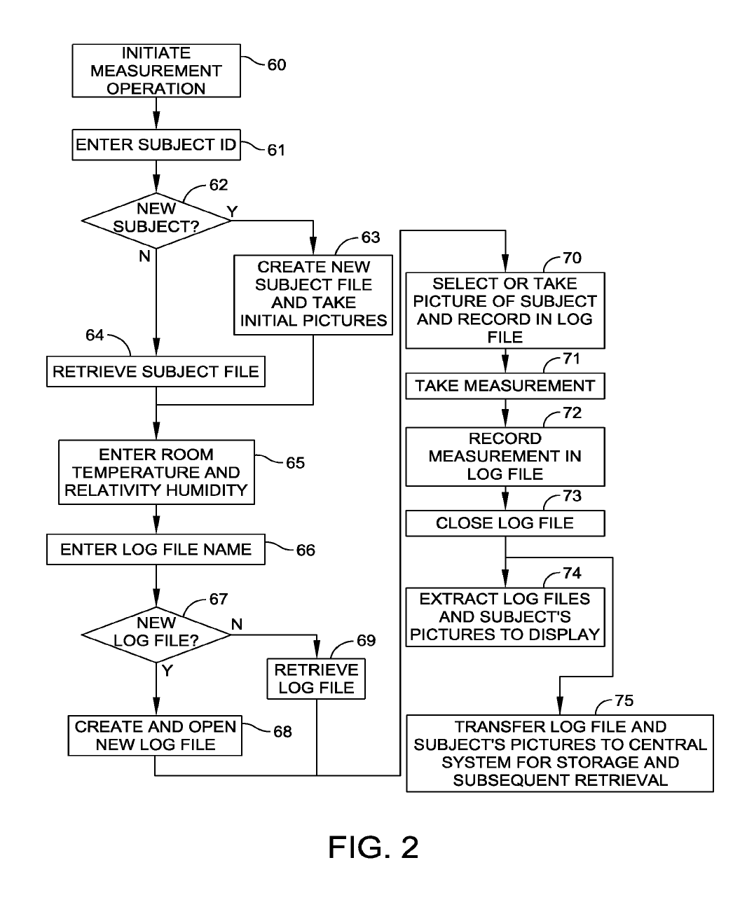

Now referring to FIGS. 1 and 2, a typical measurement session begins when a diagnostician interacts with the data processing system 40 to initiate a measurement operation in step 60 of FIG. 2. Step 60 typically includes energizing the probe 21 and the data processing system 40 and then selecting an appropriate application program (i.e., an “APP”) to be executed in the data processing system 40. At step 61 the diagnostician enters basic patient identification information through a keyboard display on the data processing system 40 using its integral data input/output screen. If the patient is new, step 63 instructs the diagnostician to enter further information to create a new subject file and enables the diagnostician to take one or more pictures, preferably with the integral camera that may include images of the patient and of the measurement area. If the patient is a previous subject, step 64 retrieves the patient's file for display and verification at the module 40.

In the disclosed sequence, step 65 enables the diagnostician to enter room temperature and relative humidity that is measured externally. Both of these parameters are important to accurate processing the measurement data. Other parameters could also be measured.

This embodiment stores measurement data in log files including time-stamped impedance measurement data. Step 66 instructs the diagnostician to enter a log file name. If there is no existing matching log file, step 68 creates a new log file for the patient that may be linked to the patient's subject file. Step 69 retrieves an existing log file. This process of steps 60 through 69 configures system 20 for making measurements.

At step 70 the diagnostician can select a prior image, such as a prior image of the measurement area and/or take another picture of the patient's measurement area. The new image is recorded in the log file for that subject. At step 71 the diagnostician takes the measurements using the probe 21 which, by virtue of the wireless communications network, is separated from any physical attachment to any other part of the system in FIG. 1. Step 72 records the measurement data in the patient's log file. When the measurement has been completed, step 73 closes the log file so it no longer can be updated.

At this point the control/applications module 43 uses step 74 to extract the log files for the patient. The diagnostician can then interact with the data processing system 40 to display the measurement data and related images, to convert the measurement data into graphical displays for a given measurement session or set of measurement sessions. This presentation facilitates an analysis of the patient's condition and any changes in that condition that have occurred over a series of measurement sessions. For example, in sessions involving wound or burn damage to a patient, the diagnostician has actual images to view rather than subjective text summaries of appearance at each measurement session.

The diagnostician can also uses step 75 to transfer the log file and patient's pictures to a central system for storage and subsequent retrieval, as by email or by a direct transfer over a wireless or other network. Thus at the end of this process the apparatus shown in FIG. 1 operating in accordance with FIG. 2 has recorded the specific measurements for a patient and stored an image of the measurement area which is correlated to the test in time. Thus by recovery of the images in sequence, a diagnostician can monitor the progress of a patient's treatment more objectively by analyzing both the measurements which can be displayed on the device as well the images without having to rely on written descriptions based on visual observations of the measurement area.

As will be apparent the specific implementation of the measurement system 20 can take many forms that are well within the purview of persons of ordinary skill in the art. FIG. 1 depicts a specific embodiment of a dermal phase meter probe 21. Other probe configurations could be substituted. Other wireless tablets, mobile telephones and like devices could be substituted. An independent imaging system could be used in lieu of, or a complement to, the imaging system provided in a wireless tablet or mobile telephone. Moreover, the foregoing description is directed to the diagnosis of patients. As known this invention can be applied to a variety of other medical and non-medical applications exist that can benefit from accurate measurements of skin hydration. For example, such measurement apparatus is useful in monitoring the progress of skin grafts. For burn patients, such measurement apparatus is useful in monitoring the rate of recovery of the stratum corneum.

This invention has been disclosed in terms of certain embodiments. It will be apparent that the foregoing and many other modifications could be made to the disclosed apparatus without departing from the invention. Therefore, it is the intent of the appended claims to cover all such variations and modifications as come within the true spirit and scope of this invention.

What is claimed is:

1. A system for measuring skin hydration comprising:
A) a probe for measuring skin impedance at a measurement site including an independently powered measurement control for obtaining measurements in response to measurement commands and for generating information including the outcome of the skin impedance measurement,
B) a portable data processing system for generating the measurement commands and for receiving measurement information generated said probe, said data processing system having an operator interface and visual display, and
C) a wireless communications network interconnecting said probe and said portable data processing system whereby skin impedance measurements can be displayed at said portable data processing system.

2. The system as recited in claim 1 wherein said portable data processing system additionally includes imaging means for imaging and displaying the measurement site.
